(19) United States
(12) Patent Application Publication
(10) Pub. No.: US 2009/0078121 A1
(43) Pub. Date: Mar. 26, 2009

(54) Air supply vent filter for air conditioning systems

(76) Inventor: Helena Hepburn, Royal Palm Beach, FL (US)

Correspondence Address: ROBERT M. DOWNEY, P.A., 6751 N. FEDERAL HWY., SUITE 300, BOCA RATON, FL, 33487, US

(21) Appl. No.: 12/284,753

(22) Filed: Sep. 25, 2008

Related U.S. Application Data
(60) Provisional application No. 60/975,457, filed on Sep. 26, 2007.

(51) Int. Cl. B01D 46/10; A61L 9/00

(52) U.S. Cl. 96/222; 55/511

(57) ABSTRACT

A filter for placement above a supply vent grill in an air conditioning supply vent includes a peripheral frame that is sized and configured to match the interior dimensions of the air vent. The filter has at least one layer of a woven stainless steel hygienic mesh cloth that is held taut within the frame. The hygienic stainless steel mesh membrane traps dirt, dust, dust mites, germs, bacteria, mold, fungus and other particles and microbes that are carried in the airflow of the air conditioning duct system. Accordingly, the cool air exiting the supply vents and into the indoor living environment is free of these unhealthy and potentially harmful microbes and airborne particles.

This non-provisional patent application is based on provisional patent application Ser. No. 60/975,457 filed on Sep. 26, 2007 upon which applicant claims the benefit under Title 35, United States Code section 119(e).

### BACKGROUND OF THE INVENTION

1. Field of the Invention

The present invention relates to air filters for air conditioning systems and, more particularly, to a filter for placement within air supply vents of a central air conditioning system in order to prevent dirt, dust, dust mites, germs, bacteria, mold, fungus and other harmful microbes and particulate from blowing out of the supply vent and into the indoor living environment.

2. Discussion of the Related Arts

A significant percentage of the world's population suffers from various respiratory disorders, such as asthma and hay fever. While many of these people might be genetically predisposed to respiratory illness, the primary culprit is almost always airborne microbes and particulate that, when inhaled, trigger an attack. A great deal of attention has been given to outdoor environmental causes, including pollens, pollutants, and wind blown dust particles from earthen materials. However, poor air quality of indoor environments is commonly the main cause of respiratory illness, particularly asthma and bronchitis.

Today most indoor spaces in homes and offices are cooled by central air conditioning systems that pull air from each room, through ducts, and to an HVAC handler unit. In the HVAC handler unit, the air is cooled by passing it over coils that are filled with a refrigerant. After cooling, the air is forced through supply ducts leading to supply vents within each room of the interior space. The cool air exits the supply vents and fills the interior living space in order to maintain a desired temperature level. When the air is pulled from the indoor living spaces and through the return ducts, the air is directed through one or more filters that trap some dust, dirt and germs before the returning air reaches the HVAC handler unit. The primary purpose of these filters is not to keep the air clean in the indoor living environment. Instead, these filters are meant to remove dust and dirt before the air reaches the evaporator coil and blower of the HVAC handler units so that the coils stay clean for efficient heat transfer. The cooled air that is sent back into the indoor spaces often contains dust, dirt and various microbes including mold, bacteria and fungus. Some of this particulate is pulled through the ducts during operation of the air conditioning system from crawl spaces, ceiling spaces and interior wall spaces that can never be cleaned. Microbes, such as mold, bacteria and fungus often grow within the ducts, particularly if moisture becomes trapped in the air supply system. Any dirt, dust, or microbes that enter or grow within the HVAC handler unit or cool air supply ducts will eventually be blown through the supply vents and into the indoor living environment. As noted above, these airborne particles and microbes are unhealthy and will cause respiratory illnesses including, but not limited to, asthma and bronchitis.

Accordingly, there remains an urgent need for a filter that is specifically adapted for placement within the air supply vents of an air conditioning system in order to remove dirt, dust, dust mites, mold, bacteria, mildew and fungus from the cool airflow prior to entering the indoor living environment.

### OBJECTS AND ADVANTAGES OF THE INVENTION

Considering the foregoing, it is a primary object and advantage to provide an affordable air filter that is easily installed within the supply vents of a central air conditioning system, and wherein the filter is structured and disposed for blocking dust, dirt, dust mites and harmful microbes so that only clean, healthy air enters the indoor living environment.

It is a further object of the present invention to provide an air filter for installation within the air supply vents of a central air conditioning system, and wherein the air filter includes a hygienic mesh material that traps dust, dirt, dust mites, and microbes including mold, bacteria, mildew and fungus.

It is still a further object of the present invention to provide an affordable filter that is easily installed within the air supply vents of an air conditioning system, and wherein the filter is structured to remove harmful particulate and microbes from the airflow, thereby greatly improving the air quality of the indoor living environment.

It is still a further object of the present invention to provide a filter for air supply vents of an air conditioning system that makes the air within an indoor living environment cleaner and safer to breathe.

It is yet a further object of the present invention to provide a filter for air conditioning supply vents that helps to relieve respiratory problems, reduce allergens and control dust mites from contaminating the air of an indoor living environment.

It is still a further object of the present invention to provide a filter for easy installation within the air supply vents of an air conditioning system, and wherein the filter can be manufactured for easy cleaning and replacement or, alternatively, for disposal.

These and other objects and advantage of the present invention are more readily apparent with reference to the detailed description and accompanying drawings.

### SUMMARY OF THE INVENTION

The present invention is directed to an air filter for placement above the grill in an air conditioning supply vent. The filter includes a peripheral frame that is sized and configured to match the interior dimensions of the air vent. The filter further includes a woven stainless steel hygienic mesh cloth that is held taut within the frame. The hygienic stainless steel mesh membrane traps dirt, dust, dust mites, germs, bacteria, mold and fungus that are carried in the airflow of the ducts of the air conditioning system. Accordingly, the cool air exiting the supply vents and into the indoor living environment is free of these unhealthy and potentially harmful microbes and airborne particles.

### BRIEF DESCRIPTION OF THE DRAWINGS

For a fuller understanding of the nature of the present invention, reference should be made to the following detailed description taken in conjunction with the accompanying drawings in which:

FIG. 1 is an exploded perspective view showing the vent grill of an air supply vent in an air conditioning system removed and the filter of the present invention being installed within the air vent, above the vent grill;

FIG. 2 is a side elevational view shown in cross section, with the filter of the present invention shown installed within the air vent and the vent grill replaced and attached to the air vent;

FIG. 3 is top perspective view showing a further embodiment of the filter of the present invention; and

FIG. 4 is a top plan view showing still a further embodiment of the present invention including air freshener strips on the frame of the filter for releasing a pleasant fragrance into the flow of cool air exiting the filter and A.C. supply vent.

Like reference numerals refer to like parts throughout the several views of the drawings.

### DETAILED DESCRIPTION OF THE PREFERRED EMBODIMENTS

Referring to the several views of the drawings, the air conditioning supply vent filter of the present invention is shown in accordance with several embodiments thereof.

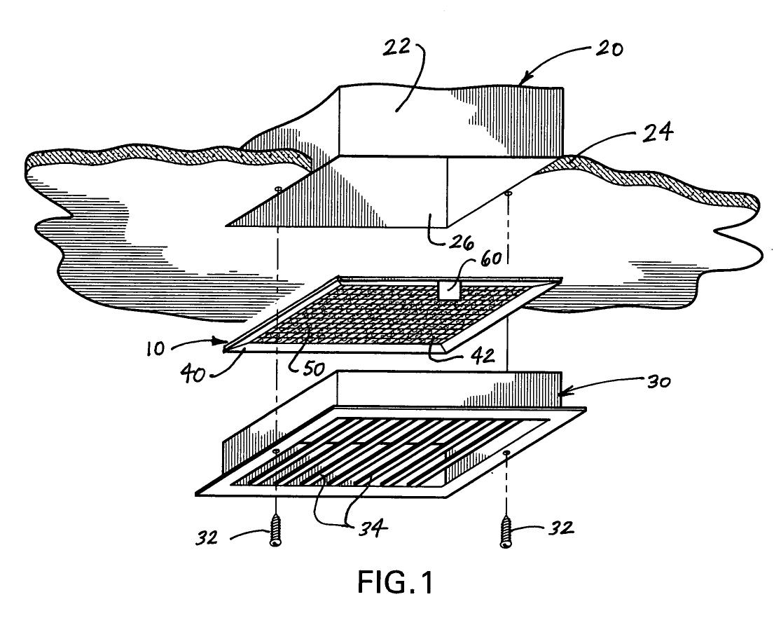

Referring initially to FIG. 1, the filter, generally indicated as 10, is shown being installed within a supply vent 20 of a central air conditioning system. The supply vent 20 typically includes a box structure 22 that is installed within the ceiling 24 or wall. The box structure 22 of the supply vent connects to an air supply duct that delivers air from the cooling unit (HVAC handler) to the one or more supply vents of the air conditioning system. The opening 26 of the supply vent box 22 is normally covered with a vent grill 30 that fastens to the vent box or surrounding structure with the use of two or more screws 32. The supply vent grill 30 usually has adjustable fins or louvers 34 that are positioned for directing the flow of cool air from the supply vent at a desired angle into the room. These fins or louvers 34 can also be adjusted to regulate cool airflow into the room by moving the fins between a fully closed position, to various partially opened positions or to a fully open position for maximum airflow, thereby controlling the room temperature relative to adjacent rooms.

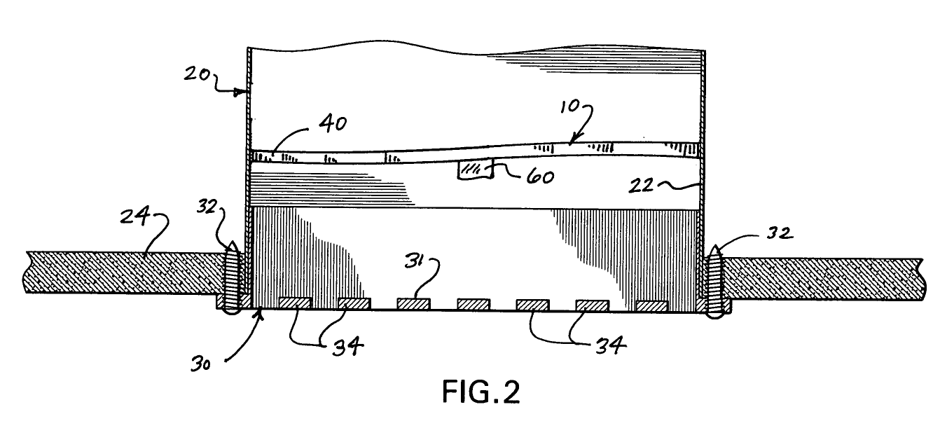

The filter 10 of the present invention is installed within the air supply vent 20, above the supply vent grill 30. As illustrated in FIG. 1, installation of the filter 10 is achieved by first removing the supply vent grill 30 from the vent box 22. The filter 10 can then be fitted into the supply vent box 22, as seen in FIG. 2. Alternatively, the filter 10 may rest on the top side 31 of the vent grill 30. In either case, the filter 10 is disposed in blocking relation to cool airflow that is directed out from the supply vent. Once the filter 10 is installed, the grill 30 is replaced, using the screws 32, as shown in FIG. 2. The air filter 10, now installed within the air vent, traps dust, dirt, dust mites, mold, bacteria, mildew, fungus, and other airborne particulate and microbes that are carried in the cool airflow. Accordingly, the cool air that exits the supply vent grill 30 is free of these unhealthy and potentially harmful particles and microbes.

The filter 10 includes a peripheral frame structure 40 that surrounds a central area 42. The frame 40 may be constructed of stainless steel, aluminum, plastic, or cardboard. The frame 40 is specifically sized and configured to fit neatly within the air supply vent box 22, matching the interior dimensions thereof, so that the filter 10 confronts airflow traveling through the vent. The filter further includes a filter membrane 50 defined by a woven stainless steel hygienic mesh material fitted within the area 42 surrounded by the frame structure. The hygienic mesh material filters membrane 50 is pulled taut within the confines of the surrounding frame structure 40 to provide a barrier that captures airborne particulate and microbes as cool air is forced through the hygienic mesh filter membrane 50.

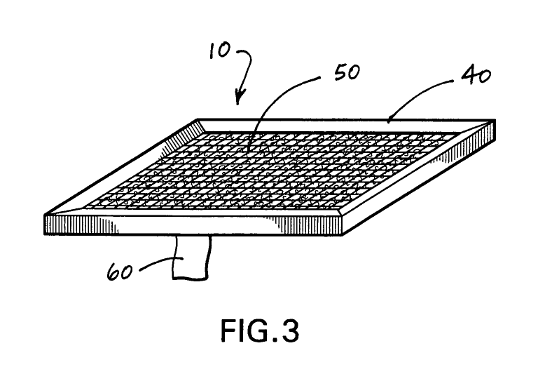

Referring to FIG. 3, the filter may be constructed to have a thickness suitable for carrying a core material, such as a HEPA (High Efficiency Particulate Air) filter, sandwiched between opposite stainless steel hygienic mesh filter membranes 50. Alternatively, as shown in FIGS. 1 and 2, the filter may be provided with a single woven stainless steel hygienic mesh membrane 50, thereby providing a thin, low profile filter that easily fits within the supply vent box 22. The single woven stainless steel hygienic mesh membrane 50 may be HEPA standard. As shown in FIGS. 1-3, the filter 10 may further include a tab 60 to facilitate ease of grasping and removal of the filter from the supply vent 20 to allow for cleaning or disposal and replacement of a clean filter 10 within the supply vent 20.

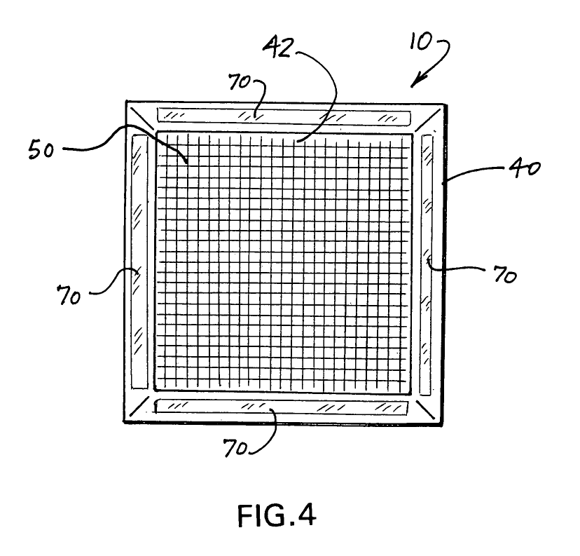

Referring to FIG. 4, a further embodiment of the filter 10 provides an air-freshener composition 70 applied to surfaces of the frame structure. The air-freshener composition 70 is specifically structured and disposed to release a pleasant fragrance into the airflow passing through the filter 10 and exiting the supply vent 20 into the surrounding environment.

While the present invention has been shown and described in accordance with several preferred and practical embodiments thereof, it is recognized that departures from the instant disclosure are fully contemplated within the spirit and scope of the present invention, which is not to be limited, except as defined in the following claims as interpreted under the Doctrine of Equivalents.

What is claimed is:

1. An air supply vent filter for air-conditioning systems comprising:
a peripheral frame surrounding a central area; and
at least one filter membrane attached to said peripheral frame and held taut throughout the central area, and said at least one filter membrane including a hygienic stainless steel mesh fabric.

2. The air supply vent filter has recited in claim 1 further comprising:
a tab extending from said peripheral frame and being structured a and disposed for grasping in order to remove the filter from an air supply vent.

3. The air supply vent filter has recited in claim 2 further comprising:
an air-freshener composition applied to said peripheral frame and formulated for releasing a fragrance into a flow of air passing through said filter.

4. The air supply vent filter has recited in claim 3 wherein said peripheral frame is formed of cardboard.

5. The air supply vent filter has recited in claim 3 wherein said peripheral frame is formed of plastic.

6. The air supply vent filter has recited in claim 3 wherein said peripheral frame is formed of stainless steel.

7. The air supply vent filter has recited in claim 3 wherein said peripheral frame is formed of aluminum.

8. An air supply vent filter for air-conditioning systems comprising:
a peripheral frame surrounding a central area;
at least one filter membrane attached to said peripheral frame and held taut throughout the central area, and said at least one filter membrane including a hygienic stainless steel mesh fabric;
a tab extending from said peripheral frame and being structured and disposed for grasping in order to remove the filter from an air supply vent; and
an air-freshener composition applied to said peripheral frame and formulated for releasing a fragrance into a flow of air passing through said filter.

9. The air supply vent filter has recited in claim 8 wherein said peripheral frame is formed of cardboard.

10. The air supply vent filter has recited in claim 8 wherein said peripheral frame is formed of plastic.

11. The air supply vent filter has recited in claim 8 wherein said peripheral frame is formed of stainless steel.

12. The air supply vent filter has recited in claim 8 wherein said peripheral frame is formed of aluminum.
